(19) United States
(12) Patent Application Publication
(10) Pub. No.: US 2021/0101002 A1
(43) Pub. Date: Apr. 8, 2021

(54) RODS WITH ELECTRICAL STIMULATION FOR PROMOTING BONE FUSION

(71) Applicant: University of Washington, Seattle, WA (US)

(72) Inventors: Rajiv Saigal, Seattle, WA (US); Samantha Kokoska, Seattle, WA (US); Alexis Fleming, Seattle, WA (US); Taylor Monroe-Jones, Seattle, WA (US); Kai-Bin Ooi, Seattle, WA (US); Alejandro Diaz, Seattle, WA (US)

(21) Appl. No.: 17/061,465

(22) Filed: Oct. 1, 2020

Related U.S. Application Data
(60) Provisional application No. 62/910,285, filed on Oct. 3, 2019.

(51) Int. Cl. A61N 1/32; A61N 1/05; A61F 2/44

(52) U.S. Cl. CPC A61N 1/326; A61F 2/4455; A61N 1/05

(57) ABSTRACT

Systems and methods for fusing vertebrae and systems and methods for repairing a bone defect employ a fixation assembly and current delivered to the fixation assembly to electrically stimulate osteogenesis in tissue surrounding the fixation assembly. An implantable system for fusing vertebrae includes a fixation assembly and an implantable stimulation unit. The fixation assembly includes an elongated rod configured to accommodate bone ingrowth. The fixation assembly is configured to structurally couple at least two of the two or more vertebrae via the elongated rod. The implantable stimulation unit is configured to supply electrical current to the elongated rod to promote bone ingrowth into the elongated rod.

### CROSS-REFERENCES TO RELATED APPLICATIONS

This application claims the benefit of U.S. Provisional Application No. 62/910,285, filed Oct. 3, 2019, the contents of which are hereby incorporated by reference in its entirety for all purposes.

### BACKGROUND

Spinal fusion surgery, where two or more vertebrae are fused together, can be performed on patients with spinal injuries, deformities, degeneration, disease, tumors, etc. Existing approaches have used rods, screws and bone grafts to fuse two or more vertebrae together. In some instances the rods are bent to match spine curvature. In some instances, however, the rods and screws employed in existing approaches may break thereby resulting in pain, discomfort and/or corrective surgery.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Implantable systems for fusing two or more vertebrae, and related methods, employ electrical stimulation of tissue surrounding a fixation assembly used to structurally couple the vertebrae to promote bone ingrowth into at least part of the fixation assembly. In many embodiments, the fixation assembly includes an elongated rod that is configured to accommodate bone ingrowth. In some embodiments, the elongated rod has a porosity configured to accommodate bone ingrowth. In other embodiments, the elongated rod has a solid core, but has surface features to promote bone growth. In many embodiments, an implantable stimulation unit supplies electrical current to the elongated rod to produce the electrical stimulation of the tissue surrounding the rod. The resulting bone ingrowth into or on the surface of the elongated rod serves to reinforce the fixation assembly, thereby inhibiting failure of the fixation assembly. In some embodiments, an electrical stimulation-screw and rod fixation system accommodates and stimulates additional bony fusion at a surgical site via additional bone growth along or inside of the rod. The electrical stimulation promotes osteogenesis. The mechanical features of the rod accommodate bone ingrowth. The implantable stimulation circuit components maintain the current and/or voltage within a desired range to promote osteogenesis.

Thus, in one aspect, an implantable system for fusing two or more vertebrae of a spinal column of a patient includes a fixation assembly and an implantable stimulation unit. The fixation assembly includes an elongated rod configured for implantation in alignment with the spinal column, to at least partially span the two or more vertebrae and to accommodate bone ingrowth. The fixation assembly is configured to structurally couple at least two of the two or more vertebrae via the elongated rod. The implantable stimulation unit is configured to supply electrical current to the elongated rod to promote bone ingrowth into the elongated rod. In some embodiments, the implantable stimulation unit includes a battery for powering the implantable stimulation unit.

In many embodiments, the fixation assembly includes a second elongated rod configured for implantation in alignment with the spinal column, to at least partially span the two or more vertebrae and to accommodate bone ingrowth. In such embodiments, the fixation assembly is configured to structurally couple at least two of the two or more vertebrae via the second elongated rod. In some embodiments, the implantable stimulation unit is configured to supply electrical current to the second elongated rod to promote bone ingrowth into the second elongated rod.

In many embodiments, the fixation assembly includes bone screws. Each of the bone screws can be configured for structurally coupling the elongated rod with one of the two or more vertebrae.

In some embodiments, the implantable stimulation unit is configured to induce flow of electrical current between the elongated rod and tissue of the patient adjacent to the elongated rod to stimulate osteogenesis. In some embodiments, the implantable stimulation unit is further configured to induce an electromagnetic field that extends through tissue of the patient adjacent to the elongated rod to stimulate osteogenesis. In some embodiments, the implantable stimulation unit generates an alternating current within the elongated rod.

In some embodiments, the implantable stimulation unit is configured to induce an electromagnetic field that extends through tissue of the patient adjacent to the elongated rod to stimulate osteogenesis. In some embodiments, the implantable stimulation unit generates an alternating current within the elongated rod.

The elongated rod can have any suitable configuration to accommodate bone ingrowth. For example, in some embodiments, the elongated rod has exterior elongated grooves configured to accommodate bone ingrowth. In some embodiments, the elongated rod defines an elongated lumen configured to accommodate bone ingrowth. In some embodiments, the elongated rod has a porous surface configured to accommodate bone ingrowth.

The elongated rod can be made using any suitable approach. For example, the elongated rod can be fabricated by three-dimensional printing.

The elongated rod can be made from any suitable material. For example, the elongated rod can include one or more of titanium, cobalt chrome, stainless steel, or other metal alloy.

In another aspect, a method of fusing two or more vertebrae of a patient includes mechanically coupling the two or more vertebrae by implanting a fixation assembly comprising an elongated rod configured to accommodate bone ingrowth, wherein the elongated rod is implanted to at least partially span the two or more vertebrae. The method further includes implanting a stimulation unit into the patient. The method further includes supplying, by the stimulation unit, electrical current to the elongated rod to promote bone ingrowth into the elongated rod. Any suitable fixation assembly and implantable stimulation unit can be used to practice the method, including those described herein.

In another aspect, an implantable system for repairing a defect of a bone of a patient includes a fixation assembly and an implantable stimulation unit. The fixation assembly includes a structural member configured to be interfaced with the bone, at least partially span the defect and to accommodate bone ingrowth. The implantable stimulation unit is configured to supply electrical current to the structural member to promote bone ingrowth into the structural member. In many embodiments, the implantable stimulation unit includes a battery for powering the implantable stimulation unit.

In some embodiments, the fixation assembly includes bone screws. Each of the bone screws can be configured for structurally coupling the structural member with the bone.

In some embodiments, the implantable stimulation unit is configured to induce flow of electrical current between the structural member and tissue of the patient adjacent to the structural member to stimulate osteogenesis. In some embodiments, the implantable stimulation unit is further configured to induce an electromagnetic field that extends through tissue of the patient adjacent to the structural member to stimulate osteogenesis. In some embodiments, the implantable stimulation unit generates an alternating current within the elongated rod.

In some embodiments, the implantable stimulation unit is configured to induce an electromagnetic field that extends through tissue of the patient adjacent to the structural member to stimulate osteogenesis. In some embodiments, the implantable stimulation unit generates an alternating current within the elongated rod.

The structural member can have any suitable configuration to accommodate bone ingrowth. For example, in some embodiments, the structural member has exterior elongated grooves configured to accommodate bone ingrowth. In some embodiments, the structural member has a porous surface configured to accommodate bone ingrowth.

The structural member can be made using any suitable approach. For example, the structural member can be fabricated by three-dimensional printing.

The structural member can be made from any suitable material. For example, the structural member can include one or more of titanium, cobalt chrome, stainless steel, or other metal alloy.

In another aspect, a method of repairing a defect of a bone of a patient includes mechanically coupling two or more portions of the bone by implanting a fixation assembly that includes a structural member configured to accommodate bone ingrowth. The structural member is implanted to at least partially span the defect. The method further includes implanting a stimulation unit into the patient. The method further includes supplying. by the stimulation unit, electrical current to the structural member to promote bone ingrowth into the structural member. Any suitable fixation assembly and implantable stimulation unit can be used to practice the method, including those described herein.

For a fuller understanding of the nature and advantages of the present invention, reference should he made to the ensuing detailed description and accompanying drawings.

### BRIEF DESCRIPTION OF THE DRAWINGS

Various embodiments in accordance with the present disclosure will be described with reference to the drawings, in which:

FIG. 1 illustrates an implantable system for fusing two or more vertebrae that employs electromagnetic stimulation and electrical current stimulation of tissue surrounding a fixation assembly to promote bone ingrowth, in accordance with embodiments.

FIG. 2 illustrates a hollow elongated rod of a fixation assembly that is configured to accommodate bone ingrowth, in accordance with embodiments.

FIG. 3 illustrates a solid elongated rod of a fixation assembly that is configured to accommodate bone ingrowth, in accordance with embodiments.

FIG. 4 is a simplified schematic diagram of an example direct current supply circuit that can be used to supply direct current to a fixation assembly to produce electrical current stimulation to tissue surrounding the fixation assembly, in accordance with embodiments.

FIG. 5 is a simplified schematic diagram of an example alternating current supply circuit that can be used to supply alternating current to a fixation assembly to produce electromagnetic stimulation to tissue surrounding the fixation assembly, in accordance with embodiments.

FIG. 6 is a simplified schematic diagram illustrating a method of fusing two or more vertebrae, in accordance with embodiments.

FIG. 7 and FIG. 8 illustrate the results of an analysis predicting column compression strength of an elongated rod employed in a vertebrae fixation assembly, in accordance with embodiments.

FIG. 9 and FIG. 10 illustrate the results of testing of beam strength of an elongated rod employed in a vertebrae fixation assembly, in accordance with embodiments.

FIG. 11 is a chart summarizing the results of a laboratory test of the effect of electrical stimulation on bone in-growth rate.

FIG. 12 illustrates an implantable system for repairing a bone defect, wherein the system employs electromagnetic stimulation and/or electrical current stimulation of tissue surrounding a structural member to promote bone ingrowth, in accordance with embodiments.

FIG. 13 is a simplified cross-sectional schematic view of a structural member interfaced with a bone illustrating generation of electromagnetic stimulation of tissue surrounding a structural member to promote bone ingrowth, in accordance with system of FIG. 12.

FIG. 14 is a simplified schematic diagram illustrating a method of repairing a bone defect, in accordance with embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Spinal Fusion Systems and Methods

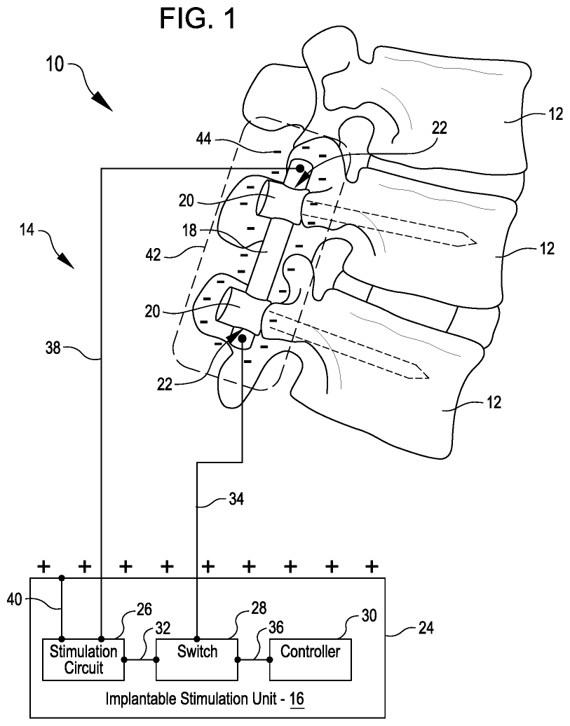

Referring now to the drawings, in which like reference numerals represent like parts throughout the several views, FIG. 1 shows an implantable system 10 for fusing two or more vertebrae 12 of a spinal column of a patient, in accordance with embodiments. The system 10 includes a fixation assembly 14 and an implantable stimulation unit 16. The fixation assembly 14 is configured to be implanted to structurally couple two or more of the vertebrae 12 so as to inhibit relative movement between the structurally coupled vertebrae 12. The implantable stimulation unit 16 is configured to supply electric current to the fixation assembly 14 to electrically stimulate tissue surrounding the fixation assembly 14 to stimulate osteogenesis and related bone ingrowth into at least a portion of the fixation assembly 14.

In the illustrated embodiment, the fixation assembly 14 includes an elongated rod 18 and bone screw assemblies 20. In many embodiments, the elongated rod 18 is formed of a suitable electrically conductive bio-compatible material. For example, the elongated rod 18 can be made from a suitable biocompatible titanium alloy. In many embodiments, the elongated rod 18 has porous exterior surface configured to accommodate ingrowth of bone into the rod 18. In many embodiments, each of the bone screw assemblies 20 is configured to be screwed into a hole drilled in one of the vertebrae 12 and has a rod aperture 22 sized to receive, interface with, and fixedly constrain a respective end portion of the rod 18. In many embodiments, the fixation assembly 14 includes two of the elongated rods 18 (and associated bone screw assemblies 20), each of which for implantation between the spinous process and respective one of the right and left transverse process.

The implantable stimulation unit 16 can have any suitable configuration for providing electrical current to the rod 18 so as to produce electrical stimulation of the tissue surrounding the rod 18 to stimulate osteogenesis and related bone ingrowth into the rod 18. For example, in the illustrated embodiments, the stimulation unit 16 includes a housing 24, a stimulation circuit 26, a switch 28, and a controller 30. In many embodiments, the stimulation circuit includes a power source (e.g., one or more battery cells) and circuit components (e.g., resistor(s), diode(s), transistor(s) etc.) for generating and supplying a suitable electrical current to the rod(s) 18. The stimulation circuit 26 is connected to a first end of the rod 18 via a current path through a first conductor 32, the switch 28 and a second conductor 34. The controller 30 controls opening and closing of the switch via a control signal transmitted to the switch over a control conductor 36. The controller 30 is configured to control the opening and closing of the switch 28 to control the supply of the current to the rod(s) 18 so that the tissue surrounding the rod(s) 18 is subjected to electrical stimulation over suitable time spans. In the illustrated embodiment, the stimulation circuit 26 is connected to a second end of the rod 18 via a second conductor 38. In the illustrated embodiment, the stimulation circuit 26 is connected to a conductive portion of the housing 24 via a conductor 40.

The system 10 can be configured to stimulate the tissue surrounding the rod 18 by generating a suitable electromagnetic field 42 that extends through the tissue and/or by inducing a suitable electrical current flow 44 (e.g., in a range from 10 μA to 10 mA) between the rod 18 and the tissue surrounding the rod 18. For example, in some embodiments, the system 10 is configured to transmit a suitable varying electrical current, such as a suitable alternating electrical current, between the first and second ends of the rod 18 so as to generate a suitable electromagnetic field 42 in the tissue surrounding the rod 18 to stimulate osteogenesis and related bone ingrowth into the rod 18. In some embodiments, the system 10 is configured to induce a suitable voltage differential (e.g., in a range of 1 mV to 10V) between the rod 18 and the housing 24 so as to induce a current flow between the rod 18 and tissue surrounding the rod 18 to stimulate osteogenesis and related bone ingrowth into the rod 18. In some embodiments, the system 10 is configured to generate both of the electromagnetic field 42 and induce the current flow 44 by both transmitting a suitable varying electrical current through the rod 18 and inducing a suitable voltage differential between the rod 18 and the housing 24.

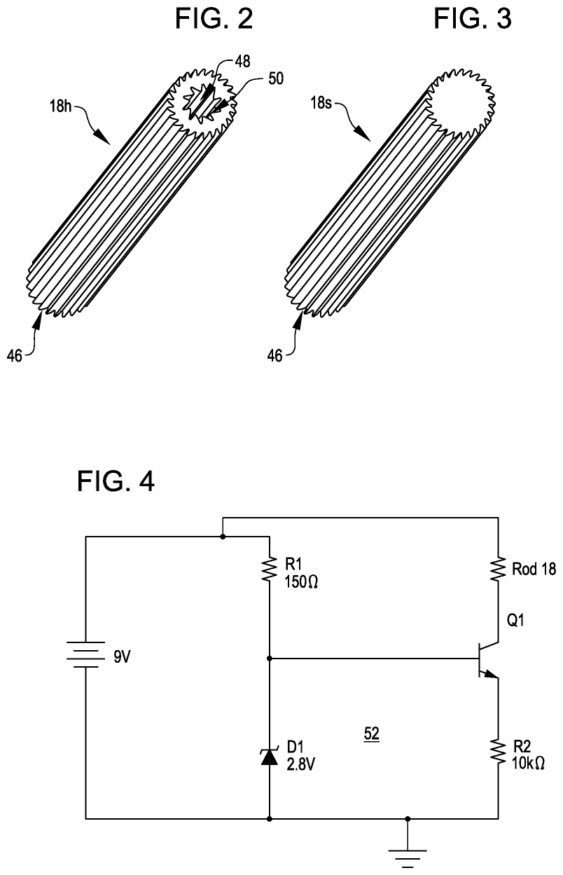

The rod 18 can have any suitable configuration that provides a suitable amount of strength and stiffness for reacting a suitable range of loading without failure and that accommodates bone ingrowth into the rod 18. For example, FIG. 2 illustrates a suitable hollow elongated rod 18h that can he used as the rod 18. The hollow rod 18h has longitudinally extending exterior grooves 46 distributed around the exterior perimeter of the hollow rod 18h. The exterior grooves 46 are shaped to accommodate bone ingrowth resulting from the osteogenesis stimulated by the electrical stimulation of the tissue surrounding the rod 18. The hollow rod 18h further defines a longitudinally extending lumen 48 configured to accommodate bone ingrowth therein. In the illustrated embodiment, the hollow rod 18h has longitudinally extending grooves 50 distributed around the lumen 48. FIG. 3 illustrates a solid elongated rod 18s that can be used as the rod 18. The solid rod 18s is configured similar to the hollow rod 18h, but without the lumen 48.

FIG. 4 is a simplified schematic diagram of an example direct current supply circuit 52 that can be included in the stimulation circuit 26 to supply direct current to the rod 18. The circuit 52 is configured to supply a constant current to the rod 18. The circuit 52 can be packaged and implanted with connection to the rod and screw system. The electrical connection may be achieved via mechanical connectors, caps, soldering, etc.

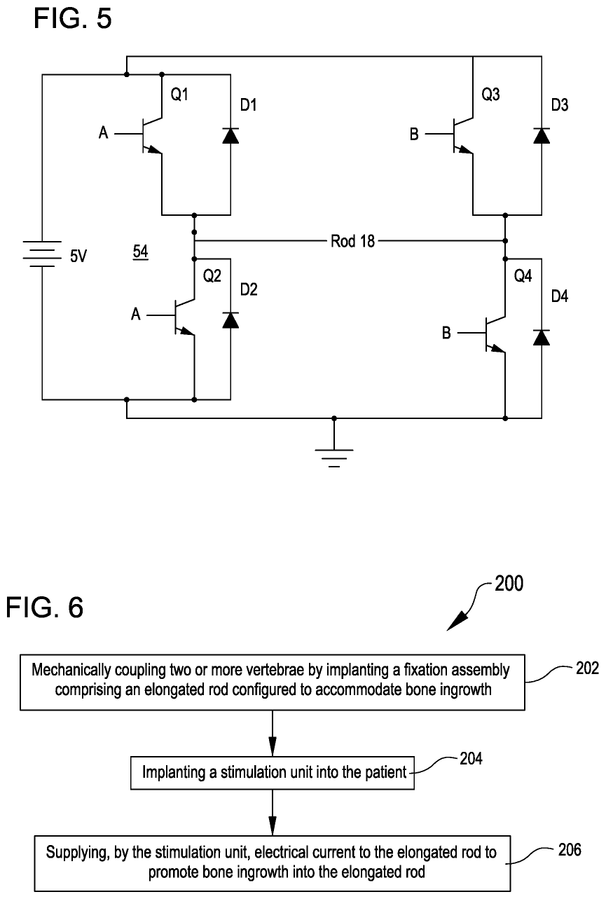

FIG. 5 is a simplified schematic diagram of an example square wave alternating current supply circuit 54 that can be used to transmit a suitable alternating current through the rod 18 to produce electromagnetic stimulation to tissue surrounding the rod 18. The current supply circuit 54 is controlled via pulse width modulation control inputs applied to pins (A), (B). The current supply circuit 54 can be packaged and implanted with connection to the rod and screw system. The electrical connection may be achieved via mechanical connectors, caps, soldering, etc.

FIG. 6 is a simplified schematic diagram illustrating a method 200 of fusing two or more vertebrae, in accordance with embodiments. The method 200 can be accomplished in conjunction with any suitable implantable system including, for example, the implantable system 10.

In act 202, two or more vertebrae of a spinal column of a patient are mechanically coupled by implanting a fixation assembly that includes an elongated rod configured to accommodate bone ingrowth. Act 202 can be accomplished by implanting the fixation assembly 14 into the patient to mechanically couple the vertebrae 12 as illustrated in FIG. 1. The fixation assembly 14 can include a second elongated rod 18 configured to accommodate bone ingrowth. The second elongated rod 18 can be implanted to at least partially span the two or more vertebrae 12. The method 200 can include generating dimensional data for the two or more vertebrae 12 and configuring the elongated rod 18 based on the dimensional data. In some embodiments of the method 200, the elongated rod 18 has exterior grooves 46 configured to accommodate bone ingrowth. In some embodiments of the method 200, the elongated rod 18 defines an elongated lumen 48 configured to accommodate bone ingrowth. In some embodiments of the method 200, the elongated rod 18 is fabricated by three-dimensional printing. In some embodiments of the method 200, the fixation assembly 14 includes bone screws 20. Each of the bone screws 20 can be used to structurally couple the elongated rod 18 with a respective one of the two or more vertebrae 12. In many embodiments of the method 200, the elongated rod 18 is fabricated of a suitable biocompatible conductive material (e.g. a suitable biocompatible titanium alloy).

In act 204, a stimulation unit is implanted into the patient. Act 204 can be accomplished by implanting the stimulation circuit 16 into the patient.

In act 206, an electrical current is supplied to the elongated rod, by the stimulation unit, to promote bone ingrowth into the elongated rod. Act 206 can be accomplished via operation of the stimulation circuit 16. In some embodiments of the method 200, the stimulation unit 16 induces flow of electrical current between the elongated rod 18 and tissue of the patient adjacent to the elongated rod 18 to stimulate osteogenesis. In some embodiments of the method 200, the stimulation unit 16 induces an electromagnetic field that extends through tissue of the patient adjacent to the elongated rod 18 to stimulate osteogenesis. In some embodiments of the method 200, the stimulation unit 16 generates an alternating current within the elongated rod 18.

Analysis and Testing

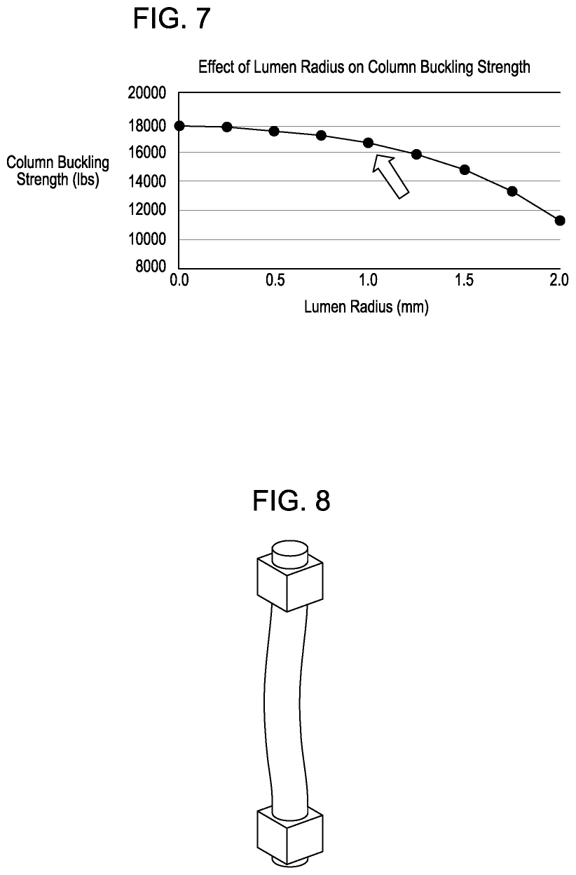

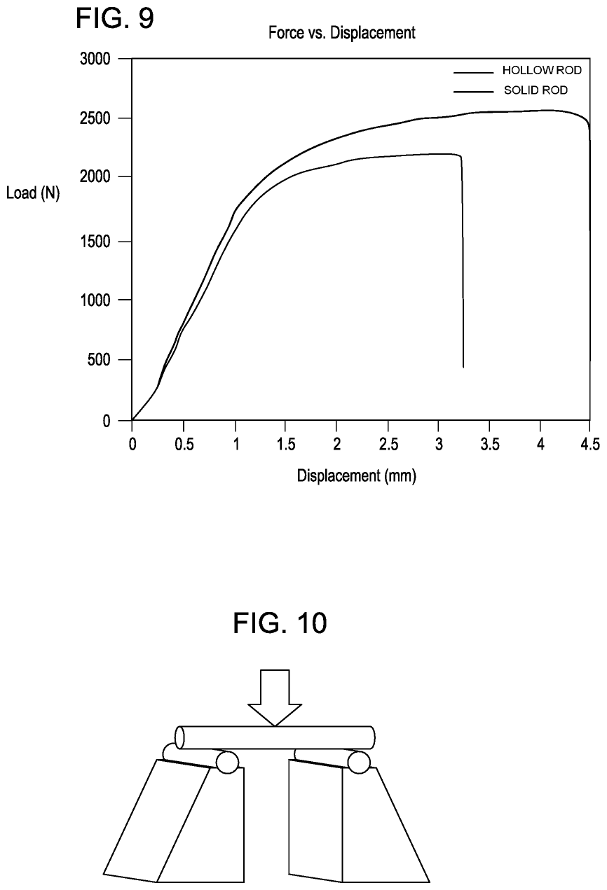

A combination of analytical assessment and testing was used to assess the strength of prototypes of the elongated rod 18. FIG. 7 and FIG. 8 illustrate the results of an analysis predicting column buckling strength of embodiments of the elongated rod 18. FIG. 9 and FIG. 10 illustrate the results of testing of beam strength of an elongated rod employed in a vertebrae fixation assembly.

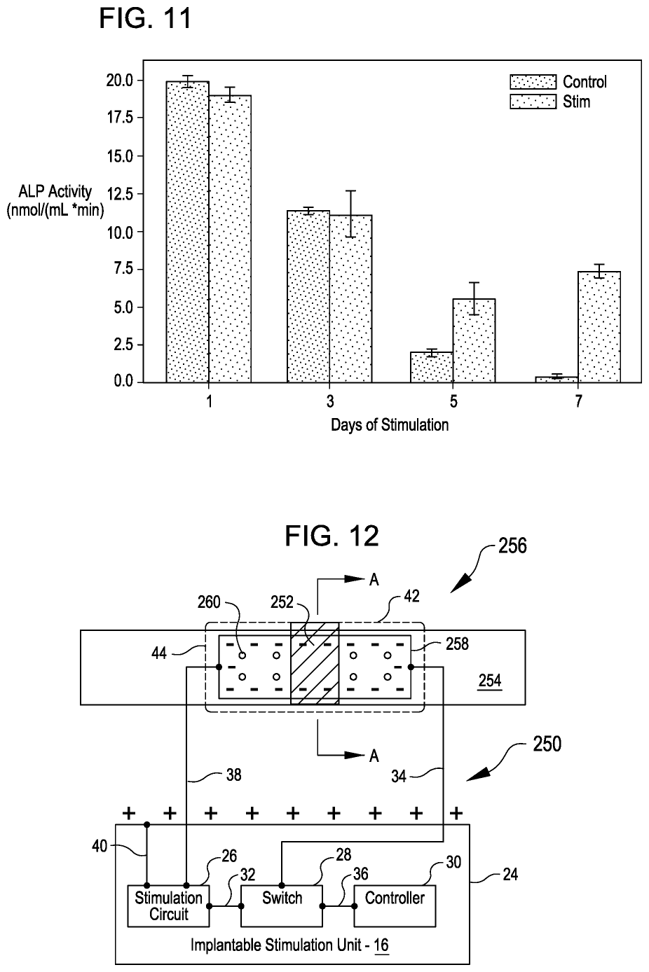

FIG. 11 is a chart summarizing the results of a laboratory test of the effect of electrical stimulation on bone in-growth rate. Cells were stimulated with 200 μA, four (4) hours per day for seven (7) days. Alkaline phosphatase activity (ALP) was used as an indicator of osteogenesis (bone fusion).

Bone Defect Repair Systems and Methods

FIG. 12 shows an implantable system 250 for repairing a defect 252 of a bone 254 of a patient, in accordance with embodiments. The system 250 includes a fixation assembly 256 and the implantable stimulation unit 16 described herein. The fixation assembly 256 is configured to be fixedly coupled with the bone 254 so as to span the defect 252 to reinforce the bone in the vicinity of the defect 252. The implantable stimulation unit 16 is configured to supply electric current to the fixation assembly 256 to electrically stimulate tissue surrounding the fixation assembly 256 to stimulate osteogenesis and related bone ingrowth into at least a portion of the fixation assembly 256. The system 250 can be used to repair any suitable defect in any suitable bone (femur, tibia, fibula, humerus, radius, ulna, clavicle, ilium, etc.).

In the illustrated embodiment, the fixation assembly 256 includes a structural member 258 and bone screws 260. In many embodiments, the structural member 258 is formed of one or more suitable bio-compatible materials. For example, the structural member 258 can include an electrically conductive portion made from a suitable biocompatible titanium alloy. In some embodiments, the structural member 258 further includes an electrically insulating portion through which a conductive path formed by the electrically conductive portion extends. In many embodiments, the structural member 258 has porous exterior surface configured to accommodate ingrowth of bone into the structural member 258. In many embodiments, each of the bone screws 260 is configured to be screwed into a respective hole through the structural member 258 and into the bone 254 to fixedly attach the structural member 258 to the bone 254.

The implantable stimulation unit 16 can have any suitable configuration for providing electrical current to the structural member 258 so as to produce electrical stimulation of the tissue surrounding the structural member 258 to stimulate osteogenesis and related bone ingrowth into the structural member 258. In the embodiment illustrated in FIG. 12, which is similar to the implantable system 10, the stimulation circuit 26 is connected to a first end of the structural member 258 via the current path through the first conductor 32, the switch 28 and the second conductor 34. The controller 30 controls opening and closing of the switch via the control signal transmitted to the switch over the control conductor 36. The controller 30 is configured to control the opening and closing of the switch 28 to control the supply of the current to the structural member 258 so that the tissue surrounding the structural member 258 is subjected to electrical stimulation over suitable time spans. In the illustrated embodiment, the stimulation circuit 26 is connected to a second end of the structural member 258 via the second conductor 38. In the illustrated embodiment, the stimulation circuit 26 is connected to the conductive portion of the housing 24 via the conductor 40.

The system 250 can be configured to stimulate the tissue surrounding the structural member 258 by generating a suitable electromagnetic field 42 that extends through the tissue and/or by inducing a suitable electrical current flow 44 between the structural member 258 and the tissue surrounding the structural member 258. For example, in some embodiments, the system 250 is configured to transmit a suitable varying electrical current, such as a suitable alternating electrical current, between the first and second ends of the structural member 258 so as to generate the electromagnetic field 42 in the tissue surrounding the structural member 258 to stimulate osteogenesis and related bone ingrowth into the structural member 258. In some embodiments, the system 250 is configured to induce a suitable voltage differential between the structural member 258 and the housing 24 so as to induce a current flow between the structural member 258 and tissue surrounding the structural member 258 to stimulate osteogenesis and related bone ingrowth into the structural 258. In some embodiments, the system 250 is configured to generate both of the electromagnetic field 42 and induce the current flow 44 by both transmitting a suitable varying electrical current through the structural member 258 and inducing a suitable voltage differential between the structural member 258 and the housing 24.

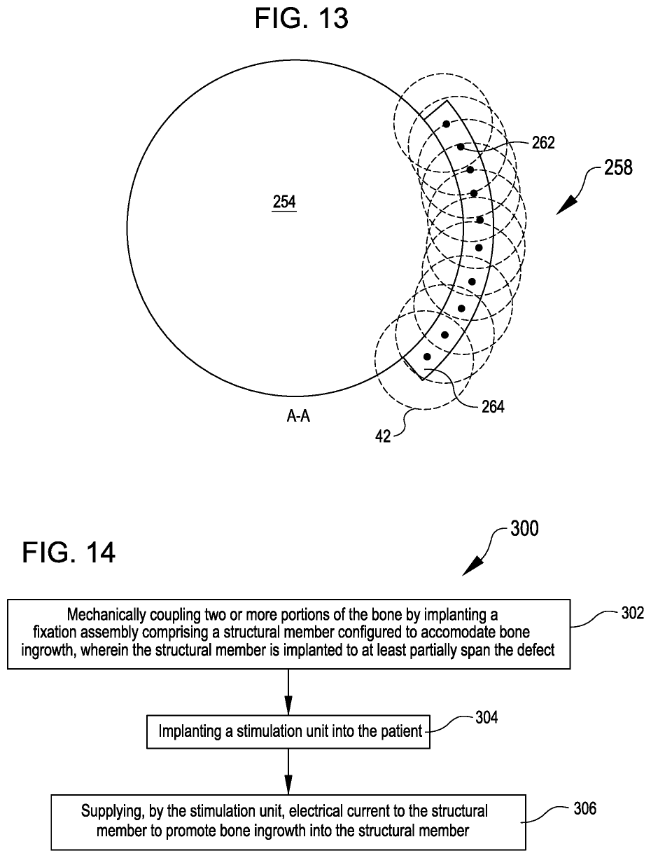

FIG. 13 is a simplified cross-sectional schematic view of an embodiment 258-1 of the structural member 258 interfaced with the bone 254. The structural member 258-1 is configured for generation of electromagnetic stimulation of tissue surrounding a structural member 258-1 to promote bone ingrowth. The structural member 258-1 include an electrically conductive portion 262, which can be made from any suitable material (e.g., biocompatible titanium alloy). The structural member 258-1 further includes an electrically insulating portion 264 through which a conductive path formed by the electrically conductive portion 262 extends. The structural member 258-1 has porous exterior surface configured to accommodate ingrowth of bone into the structural member 258-1.

FIG. 14 is a simplified schematic diagram illustrating a method 300 of repairing a defect of a bone of a patient, in accordance with embodiments. The method 300 can be accomplished in conjunction with any suitable implantable system including, for example, the implantable system 250. The method 300 can be used to repair any suitable defect in any suitable bone (femur, tibia, fibula, humerus, radius, ulna, clavicle, ilium, etc.).

In act 302, two or more portions of the bone are mechanically coupled by implanting a fixation assembly that includes a structural member configured to accommodate bone ingrowth. Act 302 can be accomplished by attaching the fixation assembly 256 to the bone 254 using any suitable approach, such as via installation of the bone screws 260. The method 300 can include generating dimensional data for the bone 254 and configuring the structural member 258 based on the dimensional data. In some embodiments of the method 300, the structural member 258 has exterior grooves configured to accommodate bone ingrowth. In some embodiments of the method 300, the structural member 258 is fabricated via an additive manufacturing approach (e.g., three-dimensional printing). In some embodiments of the method 300, the fixation assembly 256 includes bone screws 260. Each of the bone screws 260 can be used to attach the structural member 258 with the bone 254. In many embodiments of the method 300, the structural member 258 is fabricated of a suitable biocompatible conductive material (e.g. a suitable biocompatible titanium alloy).

In act 304, a stimulation unit is implanted into the patient. Act 304 can be accomplished by implanting the stimulation circuit 16 into the patient.

In act 306, an electrical current is supplied to the structural member, by the stimulation unit, to promote bone ingrowth into the structural member. Act 306 can be accomplished via operation of the stimulation circuit 16. In some embodiments of the method 300, the stimulation unit 16 induces flow of electrical current between the structural member 258 and tissue of the patient adjacent to the structural member 258 to stimulate osteogenesis. In some embodiments of the method 300, the stimulation unit 16 induces an electromagnetic field that extends through tissue of the patient adjacent to the structural member 258 to stimulate osteogenesis. In some embodiments of the method 300, the stimulation unit 16 generates an alternating current within the structural member 258.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions, and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to he understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. An implantable system for fusing two or more vertebrae of a spinal column of a patient, the implantable system comprising:
a fixation assembly comprising an elongated rod configured for implantation in alignment with the spinal column, to at least partially span the two or more vertebrae and to accommodate bone ingrowth, wherein the fixation assembly is configured to structurally couple at least two of the two or more vertebrae via the elongated rod; and
an implantable stimulation unit configured to supply electrical current to the elongated rod to promote bone ingrowth into the elongated rod.

2. The implantable system of claim 1, wherein the fixation assembly comprises a second elongated rod configured for implantation in alignment with the spinal column, to at least partially span the two or more vertebrae and to accommodate bone ingrowth, wherein the fixation assembly is configured to structurally couple at least two of the two or more vertebrae via the second elongated rod, wherein the implantable stimulation unit is configured to supply electrical current to the second elongated rod to promote bone ingrowth into the second elongated rod.

3. The implantable system of claim 1, wherein:
the fixation assembly comprises bone screws; and
each of the bone screws is configured for structurally coupling the elongated rod with one of the two or more vertebrae.

4. The implantable system of claim 1, wherein the implantable stimulation unit is configured to induce flow of electrical current between the elongated rod and tissue of the patient adjacent to the elongated rod to stimulate osteogenesis.

5. The implantable system of claim 4, wherein the implantable stimulation unit is configured to induce an electromagnetic field that extends through tissue of the patient adjacent to the elongated rod to stimulate osteogenesis.

6. The implantable system of claim 5, wherein the implantable stimulation unit generates an alternating current within the elongated rod.

7. The implantable system of claim 1, wherein the implantable stimulation unit is configured to induce an electromagnetic field that extends through tissue of the patient adjacent to the elongated rod to stimulate osteogenesis.

8. The implantable system of claim 7, wherein the implantable stimulation unit is configured to generate an alternating current within the elongated rod.

9. The implantable system of claim 1, wherein the elongated rod has exterior elongated grooves configured to accommodate bone ingrowth.

10. The implantable system of claim 9, wherein the elongated rod defines an elongated lumen configured to accommodate bone ingrowth.

11. The implantable system of claim 1, wherein the elongated rod is fabricated by three-dimensional printing.

12. The implantable system of claim 1, wherein the elongated rod comprises one or more of titanium, cobalt chrome, stainless steel, or other metal alloy.

13. The implantable system of claim 1, wherein the implantable stimulation unit comprises a battery for powering the implantable stimulation unit.

14. The implantable system of claim 1, wherein the elongated rod has a porous surface configured to accommodate bone ingrowth.

15. A method of fusing two or more vertebrae of a patient, the method comprising:
mechanically coupling the two or more vertebrae by implanting a fixation assembly comprising an elongated rod configured to accommodate bone ingrowth, wherein the elongated rod is implanted to at least partially span the two or more vertebrae;
implanting a stimulation unit into the patient; and
supplying, by the stimulation unit, electrical current to the elongated rod to promote bone ingrowth into the elongated rod.

16. The method of claim 15, wherein:
the fixation assembly comprises a second elongated rod configured to accommodate bone ingrowth, wherein the second elongated rod is implanted to at least partially span the two or more vertebrae; and
the method further comprises supplying, by the stimulation unit, electrical current to the second elongated rod to promote bone ingrowth into the second elongated rod.

17. The method of claim 15, further comprising:
generating dimensional data for the two or more vertebrae; and
configuring the elongated rod based on the dimensional data.

18. The method of claim 15, wherein the elongated rod has exterior grooves configured to accommodate bone ingrowth.

19. The method of claim 18, wherein the elongated rod defines an elongated lumen configured to accommodate bone ingrowth.

20. The method of claim 15, wherein the elongated rod is fabricated by three-dimensional printing.

21. The method of claim 15, wherein:
the fixation assembly comprises bone screws; and
each of the bone screws structurally couples the elongated rod with one of the two or more vertebrae.

22. The method of claim 15, wherein the stimulation unit induces flow of electrical current between the elongated rod and tissue of the patient adjacent to the elongated rod to stimulate osteogenesis.

23. The method of claim 22, wherein the stimulation unit induces an electromagnetic field that extends through tissue of the patient adjacent to the elongated rod to stimulate osteogenesis.

24. The method of claim 23, wherein the stimulation unit generates an alternating current within the elongated rod.

25. The method of claim 15, wherein the stimulation unit induces an electromagnetic field that extends through tissue of the patient adjacent to the elongated rod to stimulate osteogenesis.

26. The method of claim 25, wherein the stimulation unit generates an alternating current within the elongated rod.

27. The method of claim 15, wherein the elongated rod comprises one or more of titanium, cobalt chrome, stainless steel, or other metal alloy.

28. The method of claim 15, wherein the stimulation unit comprises a battery for powering the stimulation unit.

29. The method of claim 15, wherein the elongated rod has a porous surface configured to accommodate bone ingrowth.

30.-55. (canceled)
